(19) United States
(12) Patent Application Publication
(10) Pub. No.: US 2022/0331368 A1
(43) Pub. Date: Oct. 20, 2022

(54) PHARMACEUTICAL COMPOSITION COMPRISING GLIA-LIKE CELLS INDUCED FROM LATE-PASSAGE HUMAN MESENCHYMAL STEM CELLS AS ACTIVE INGREDIENT FOR TREATMENT OF STROKE

(71) Applicants: SEOUL NATIONAL UNIVERSITY R&DB FOUNDATION, Seoul (KR); IUCF-HYU (INDUSTRY-UNIVERSITY COOPERATION FOUNDATION HANYANG UNIVERSITY), Seoul (KR)

(72) Inventors: Mi-Sook Chang, Seoul (KR); Seong-Ho Koh, Seoul (KR)

(73) Assignees: SEOUL NATIONAL UNIVERSITY R&DB FOUNDATION, Seoul (KR); IUCF-HYU (INDUSTRY-UNIVERSITY COOPERATION FOUNDATION HANYANG UNIVERSITY), Seoul (KR)

(21) Appl. No.: 17/615,570

(22) PCT Filed: Aug. 24, 2020
(86) PCT No.: PCT/KR2020/011244

§ 371 Date: Nov. 30, 2021

(30) Foreign Application Priority Data
Aug. 22, 2019 (KR) 10-2019-0103215

(51) Int. Cl. A61K 35/30; A61K 35/28; A61P 9/10

(52) U.S. Cl. CPC A61K 35/30; A61K 35/28; A61P 9/10

(57) ABSTRACT

The present invention relates to a pharmaceutical composition comprising glia-like cells differentiated from human mesenchymal stem cells as an active ingredient for treatment of stroke. Specifically, as a result of injecting the glia-like cells differentiated from human mesenchymal stem cells (ghMSCs) of the present invention to cerebral infarction-induced animal models, the infarct volume remarkably decreased by 50% or more and neural functions were remarkably improved, compared to a control group and a group treated with human mesenchymal stem cells (hMSCs), demonstrating that ischemic stroke (infarction) is treated by the Akt pathway of IGFBP-4 via IGF-1R. Thus, the differentiated glia-like cells of the present invention can be advantageously used as a cell therapy product for stroke.

### BACKGROUND OF THE INVENTION

### 1. Field of the Invention

The present invention relates to a pharmaceutical composition comprising late-passage human mesenchymal stem cells induced into glia-like cells (ghMSCs) as an active ingredient for treatment of stroke.

### 2. Description of the Related Art

Stroke refers to a neurological symptom that occurs when a blood vessel supplying blood to a part of the brain is blocked (infarction) or burst (cerebral hemorrhage), resulting in damage to the nervous system of the part. Stroke can be divided into two main types; the first is the blockage of blood vessels, which damages a part of the brain that was supplied with blood by the blood vessels, called infarction. It is also called ischemic stroke or cerebral infarction. The second is a rupture of a blood vessel in the brain, which causes blood to pool in the brain and damages that part of the brain. This is called hemorrhage or hemorrhagic stroke. It is known that ischemic stroke is more common than hemorrhagic stroke by about 85%. Stroke is a neurological disease caused by damage to the nervous system and is a very difficult disease to treat.

In order to treat stroke, it is important to restore the damaged nervous system. However, neurons, which are nerve cells, cannot be restored once damaged, so the function of glial cells must be restored. Among glial cells, there is a report that astrocytes that secrete growth factors to improve the microenvironment are the most helpful for treatment of stroke.

The nervous system consists of neurons and neuroglial cells. Neuroglial cells are cells that support the nervous system and constitute about 90% of the nervous system, supply substances necessary for neurons, and maintain homeostasis for a suitable chemical environment. Unlike neurons that transmit information, neuroglial cells do not have the ability to transmit information, and unlike neurons, they can be recovered after damage. Therefore, cancer that occurs in the brain occurs in glial cells, not neurons. Glial cells are the most distributed cells in the brain. The size of glial cells is about a tenth of that of nerve cells, but it is about ten times the number, and it is estimated that there will be hundreds of billions of cells. Glial cells present in the central nervous system include astrocytes that maintain blood-brain barrier, absorb glucose from blood and supply it to neurons, and help tissue regeneration to improve microenvironment; oligodendrocytes that form myelin sheath of the central nervous system; and microglia and radial glia that serve as immune cells of the central nervous system. Glial cells present in the peripheral nervous system include Schwann cells that form myelin sheath of the peripheral nervous system and satellite cells that supply nutrients to neurons.

Stem cells are considered as a promising treatment for intractable neurological diseases such as stroke because of their ability to replace damaged or lost cells in the nervous system. It has been reported that embryonic stem cells (ESCs) and induced pluripotent stem cells (iPSCs) can differentiate into various neurons and replace the damaged nervous system. However, clinical trials have not been made because of the problem that these cells can cause unwanted cancer. Therefore, ongoing clinical trials are using adult stem cells, particularly early-passage human mesenchymal stem cells (hMSCs).

Mesenchymal stem cells (MSCs) are capable of self-renewal while maintaining growth in the laboratory, and can be differentiated into various types of cells (Bianco et al., 2001; Pittenger et al., 1999; Prockop, 1997). In addition, MSCs have the potential to trans-differentiate into various neuron-like cells with neuronal activity (Munoz-Elias et al., 2003; Sanchez-Ramos et al., 2000; Trzaska et al., 2007; Woodbury et al., 2000). Human mesenchymal stem cells (hMSCs) are being studied as a cell therapy product to treat the damaged nervous system due to their high plasticity and low immune rejection properties (Thuret et al., 2006). The present inventors demonstrated that transplanted hMSCs increase the growth of damaged nerve fibers and the survival of spinal cord cells in an ex vivo spinal cord injury model (Cho et al., 2009).

Mesenchymal stem cells of adult stem cells include early-passage hMSCs and late-passage hMSCs (10 passages or more). Late-passage hMSCs secrete less growth factors or cytokines, resulting in decreased paracrine effect and decreased nervous system recovery. Therefore, early-passage hMSCs are used for the treatment of the damaged nervous system, but in the initial stage of culture, the amount of hMSCs that can be obtained is very limited, so it is difficult to obtain a sufficient amount of cells for treatment.

Thus, the present inventors induced hMSCs into glia-like cells to have the characteristics of neuroglial cells in order to solve the above problems. The present inventors confirmed that the hMSCs induced into glia-like cells (glia-like cells induced from hMSCs, hereinafter abbreviated as ghMSCs) secrete a large amount of growth factors and cytokines, thereby enhancing the paracrine effect and thus exhibiting an excellent effect in stroke treatment.

As a result of injecting the glia-like cells induced from human mesenchymal stem cells to infarction-induced brain of animal models, the infarct volume remarkably decreased by 50% or more and neural functions were remarkably improved, compared to a control group and a group treated with human mesenchymal stem cells (hMSCs), demonstrating that ischemic stroke (infarction) was treated by the Akt pathway of IGFBP-4 via IGF-1R. The present inventors completed the present invention by confirming the above results.

### SUMMARY OF THE INVENTION

It is an object of the present invention to provide a pharmaceutical composition comprising late-passage human mesenchymal stem cells induced into glia-like cells as an active ingredient for treatment of stroke.

It is another object of the present invention to provide a method for treating stroke by administering an effective amount of late-passage human mesenchymal stem cells induced into glia-like cells to a subject.

It is another object of the present invention to provide a method for producing late-passage human mesenchymal stem cells induced into glia-like cells.

To achieve the above objects, the present invention provides a pharmaceutical composition comprising late-passage human mesenchymal stem cells induced into glia-like cells as an active ingredient for treatment of stroke.

The present invention also provides a method for treating stroke by administering an effective amount of late-passage human mesenchymal stem cells induced into glia-like cells to a subject.

In addition, the present invention provides a method for producing late-passage human mesenchymal stem cells induced into glia-like cells.

### Advantageous Effect

The present invention relates to a pharmaceutical composition comprising late-passage human mesenchymal stem cells induced into glia-like cells (ghMSCs) as an active ingredient for treatment of stroke. Specifically, as a result of injecting the glia-like cells differentiated from human mesenchymal stem cells (ghMSCs) of the present invention to cerebral infarction-induced animal models, the infarct volume remarkably decreased by 50% or more and neural functions were remarkably improved, compared to a control group and a group treated with human mesenchymal stem cells (hMSCs), demonstrating that ischemic stroke (infarction) was treated by the Akt pathway of IGFBP-4 via IGF-1R. Thus, the differentiated glia-like cells of the present invention can be advantageously used as a cell therapy product for stroke.

### BRIEF DESCRIPTION OF THE DRAWINGS

FIG. 1a is a set of photographs showing the morphological changes of glia-like cells in the ghMSC induction process confirmed by bright-field images.

FIG. 1b is a set of graphs showing the expression levels of the astrocyte markers GFAP, S100β, SLC1A3 and SLC1A2; the oligodendrocyte marker Sox10; the oligodendrocyte progenitor cell marker PDGFRα; and PAPP-A, the marker that plays an important role in ischemic conditions, in human mesenchymal stem cells (hMSCs) and glia-like cells derived therefrom (ghMSCs).

FIG. 1c is a set of photographs showing the expressions of the astrocyte markers GFAP and S100β, confirmed by immunohistochemical staining.

FIG. 1d is a diagram showing the expression ratio of the astrocyte markers GFAP and S100β in all glia-like cells, respectively.

FIG. 2a is a diagram showing the increase in cell viability when treated with glia-like cells in oxygen-glucose deprivation (OGD).

FIG. 2b is a graph showing that the mRNA expression level of IGFBP-4 is increased more than 3 times in glia-like cells than in human mesenchymal stem cells.

FIG. 2c is a graph showing that the expression level of IGFBP-4 protein is increased in glia-like cells than in human mesenchymal stem cells.

FIG. 2d is a graph showing that treatment of the concentration-dependent recombinant human IGFBP-4 does not affect on cell viability.

FIG. 2e is a graph showing the cell viability measured by treating human IGFBP-4 before and after OGD treatment.

FIG. 2f is a set of graphs showing that the mechanism of the neuroprotective effect of glia-like cells is related to the PI3K/Akt pathway.

FIG. 2g is a graph showing that the mechanism of the neuroprotective effect of glia-like cells is related to the IGF1R pathway.

FIG. 3a is a diagram showing that the phosphorylation of Akt was significantly increased when the conditioned medium of glia-like cells (ghMSC-CM) was treated.

FIG. 3b is a diagram showing that the significantly increased phosphorylated Akt by the treatment of the conditioned medium of glia-like cells (ghMSC-CM) was significantly reduced by anti-BP-4.

FIG. 3c is a diagram showing that the expression of the phosphorylated Akt increased by the conditioned medium of glia-like cells (ghMSC-CM) was decreased when PPP, an IGF-1R inhibitor, was treated in the conditioned medium of glia-like cells (ghMSC-CM).

FIG. 3d is a diagram showing that the increased expression of Bax in mitochondria was reduced when the conditioned medium of glia-like cells (ghMSC-CM) was treated, indicating that the expression of Bax in mitochondria was increased again due to the inhibition of IGFBP-4 by anti-BP-4, the inhibition of Akt by wortmannin, and the inhibition of IGF-1R by PPP.

FIG. 3e is a diagram showing that the decreased expression of Bax in cytoplasm was increased when the conditioned medium of glia-like cells (ghMSC-CM) was treated, indicating that the expression of Bax in cytoplasm was reduced again due to the inhibition of IGFBP-4 by anti-BP-4, the inhibition of Akt by wortmannin, and the inhibition of IGF-1R by PPP.

FIG. 3f is a set of graphs showing that the OGD treatment did not change the expression of extracellular IGF-1 and IGF-2, but the treatment of conditioned medium of glia-like cells (ghMSC-CM) after the OGD treatment significantly increased the expression of extracellular IGF-1 compared to the control group and OGD-treated group, and this increase was reversed by the anti-IGFBP-4 antibody treatment.

FIG. 4a is a schematic diagram of a study to confirm the therapeutic effect of glia-like cells on infarction.

FIG. 4b is a set of photographs and a graph showing that the infarct volume was significantly reduced by more than 50% as a result of injection of glia-like cells (ghMSCs) 1 day after the induction of infarction, and the effect of glia-like cells (ghMSCs) was about 20% higher than that of human mesenchymal stem cells (hMSCs), indicating this effect was almost completely blocked by anti-BP-4 antibody.

FIG. 4c is a set of graphs showing that the neurological function was improved when infarction was treated using glia-like cells (ghMSCs), and this effect was disappeared when treated with anti-BP-4 antibody.

FIGS. 5a to 5f are diagrams showing that the phosphorylated Akt (pAkt) and Bcl-xl were significantly increased in the brain injected with glia-like cells, and cytochrome c, Bax and cleaved caspase-3 were significantly decreased, and these changes were significantly reduced by anti-BP-4 antibody.

FIG. 6 is a set of photographs and graphs showing that the expressions of NeuN (neuronal marker) and pAkt were increased in the brain transplanted with glia-like cells, and Bax and cleaved caspase-9 were decreased according to the injection of glia-like cells, and this effect was reduced when injected with anti-BP-4 antibody.

FIG. 7 is a schematic diagram showing the mechanism of neuronal protection of glia-like cells (ghMSCs) through IGFBP-4.

### DESCRIPTION OF THE PREFERRED EMBODIMENTS

The present invention provides a pharmaceutical composition comprising late-passage human mesenchymal stem cells (ghMSCs) induced into glia-like cells as an active ingredient for treatment of stroke.

The present invention also provides a method for treating stroke by administering an effective amount of late-passage human mesenchymal stem cells (ghMSCs) induced into glia-like cells to a subject.

There is a report that the use of neurons or glial cells differentiated from hMSCs can be used as a neuroconstructive approach for neurological disorders. In particular, the paracrine activity of hMSCs is considered to have a significant impact on clinical trials. However, most clinical trials have only used early-passage hMSCs lower than passage 5, despite the high cost, because the paracrine activity of late-passage cells is remarkably low. That is, as confirmed by the present inventors, transplantation of late-passage hMSCs from passages 11 to 14 had little effect on the behavioral recovery of stroke-induced mice.

The present inventors obtained ghMSCs with increased paracrine activity by inducing differentiation of late-passage hMSCs with low neuronal function recovery effect into glia-like cells. GhMSCs significantly protected primary culture cortical neurons damaged by oxygen-glucose deprivation (OGD). In in vivo experiments, the brain of the mouse model was significantly protected from infarction by ghMSC and the neurobehavioral function was remarkably restored by the ghMSC transplantation. In addition, the Akt signaling pathway components were activated, the antiapoptotic pathway in the brain was enhanced by the ghMSC transplantation, and the stroke protective effect was blocked by anti-BP-4. These results show that the ghMSCs that are derived from late-passage hMSCs and release IGBP-4 are a desirable cell source for treating stroke. In addition, the present inventors revealed for the first time that IGFBP-4 released from ghMSCs exhibits neuroprotective effects in in vitro and in vivo ischemic stroke models. IGFBP-4, which is highly expressed in ghMSCs compared to hMSCs, improved survival of cortical neurons damaged by oxygen-glucose deprivation (OGD) and reduced infarct volume. In addition, it was confirmed that IGBP-4 enhanced the phosphorylation of Akt through IGF-1R signaling.

The glia-like cells are at least one selected from the group consisting of oligodendroglia, astrocytes, microglia, and radial glia.

The stroke is ischemic stroke or hemorrhagic stroke.

The ghMSCs are differentiated from late-passage human mesenchymal stem cells.

The ghMSCs release IGFBP-4 (insulin-like growth factor binding protein-4).

The ghMSCs treat stroke by activating the PI3K/Akt pathway of IGFBP-4 via IGF-1R.

The effective dose of the composition is 10³˜10⁹ cells/kg, preferably 10⁴˜10⁸ cells/kg, and more preferably 6×10⁵˜6×10⁷ cells/kg. It can be administered 2˜3 times a day.

The pharmaceutical composition according to the present invention can contain 10˜95 weight % of glia-like cells differentiated from human mesenchymal stem cells based on the total weight of the composition. In addition, the pharmaceutical composition of the present invention can further include at least one active ingredient exhibiting the same or similar function in addition to the active ingredient.

The glia-like cells differentiated from human mesenchymal stem cells of the present invention can be presented as a pharmaceutical composition for treatment. Such a pharmaceutical composition can include a physiologically acceptable matrix or physiologically acceptable excipient in addition to the cells. The type of matrix and/or excipient depends on others according to the intended route of administration. The pharmaceutical composition can also optionally contain other suitable excipients or active ingredients used together in the treatment with stem cells.

In addition, the dosage of the composition can be increased or decreased depending on the route of administration, the degree of disease, the patient's gender, body weight, and age, etc. Therefore, the above dosage does not limit the scope of the present invention in any way.

The term “administration” in the present invention means introducing a predetermined substance into a patient by an appropriate method, and the composition can be administered through any general route as long as it can reach a target tissue. The route can be intraperitoneal administration, intravenous administration, intramuscular administration, subcutaneous administration, intradermal administration, oral administration, topical administration, intranasal administration, intrapulmonary administration, or rectal administration, but not always limited thereto.

In addition, the pharmaceutical composition of the present invention can be administered by any device capable of transporting an active substance to target cells. Preferred administration ways and formulations are intravenous injections, subcutaneous injections, intradermal injections, intramuscular injections, drip injections, and the like. Injections can be prepared using aqueous solvents such as physiological saline and Ringer's solution, vegetable oil, higher fatty acid esters (eg, ethyl oleate), and non-aqueous solvents such as alcohols (eg, ethanol, benzyl alcohol, propylene glycol, glycerin, etc.), and can include pharmaceutical carriers such as stabilizers for preventing deterioration (eg, ascorbic acid, sodium hydrogen sulfite, sodium pyrosulfite, BHA, tocopherol, EDTA, etc.), emulsifiers, buffers for pH adjustment, and preservatives for inhibiting the growth of microorganisms (eg, phenylmercuric nitrate, thimerosal, benzalkonium chloride, phenol, cresol, benzyl alcohol, etc.).

The present invention provides a method for producing glia-like cells differentiated from human mesenchymal stem cells.

The method for producing glia-like cells differentiated from human mesenchymal stem cells comprises the following steps:

1) obtaining late-passage human mesenchymal stem cells by culturing human mesenchymal stem cells;

2) primary culturing the late-passage human mesenchymal stem cells obtained in step 1) for 22˜26 hours in DMEM containing 10% FBS and 1 mM β-mercaptoethanol;

3) secondary culturing the late-passage human mesenchymal stem cells for 68˜76 hours in DMEM containing 10% FBS and 0.28 μg/ml of tretinoin (all-trans-retinoic acid); and

4) tertiary culturing the late-passage human mesenchymal stem cells for 8 days in DMEM containing 10% FBS, 10 ng/ml of bFGF (basic Fibroblast Growth factor), 5 ng/ml of PDGF-AA, 10 μM Forskolin and 200 ng/ml of HRG-β1 (Heregulin-β1).

Frozen vials were prepared by putting the glia-like cells differentiated from human mesenchymal stem cells, FBS stock solution and DMSO in DMEM containing fetal bovine serum (FBS), and the prepared frozen vials were placed in a freezing container containing isopropanol. After overnight storage in a −80° C. DEEP freezer, the prepared frozen vials were placed in a liquid nitrogen tank (LN2 tank) and frozen.

The frozen glia-like cells differentiated from human mesenchymal stem cells were thawed in a 37° C. water bath, and the cells in a frozen vial with a little ice were mixed well using a pipette in a biosafety hood. The cells were mixed with DMEM containing fetal bovine serum (FBS) and placed in a conical tube, and the cell pellet precipitated by centrifugation was mixed with DMEM containing fetal bovine serum (FBS). The cells were mixed with trypan blue and smeared on a cell culture dish for use.

The human mesenchymal stem cells can be adult stem cells derived from bone marrow, adipose tissue, blood, umbilical cord blood, liver, skin, gastrointestinal tract, placenta or uterus. More preferably, the human mesenchymal stem cells are bone marrow-derived mesenchymal stem cells.

The glia-like cells can be at least one selected from the group consisting of oligodendroglia, astrocytes, microglia, and radial glia.

In fact, analysis of the ghMSCs obtained in the present invention showed that 60 to 70% of ghMSCs were astrocytes, 10 to 20% were differentiated or other glial cells, and about 10% were undifferentiated hMSCs.

Since astrocytes are glial cells having the greatest paracrine effect, the presence of a large number of astrocytes means that the activity to promote the regeneration and recovery of surrounding tissues is high, indicating that it is effective in stroke treatment.

Therefore, it can be seen that the preparation method of the present invention is an optimized method for increasing the efficacy of late-passage hMSCs.

The composition is characterized in that it is administered for the treatment or amelioration of a lesion in a subject having stroke.

The present invention provides a composition for intra-individual IGFBP-4 (insulin-like growth factor binding protein-4) delivery containing glia-like cells differentiated from human bone marrow-derived mesenchymal stem cells as an active ingredient.

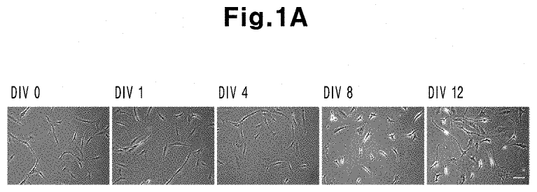

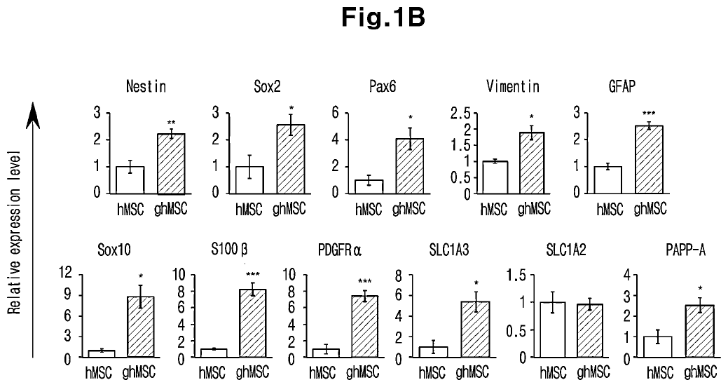

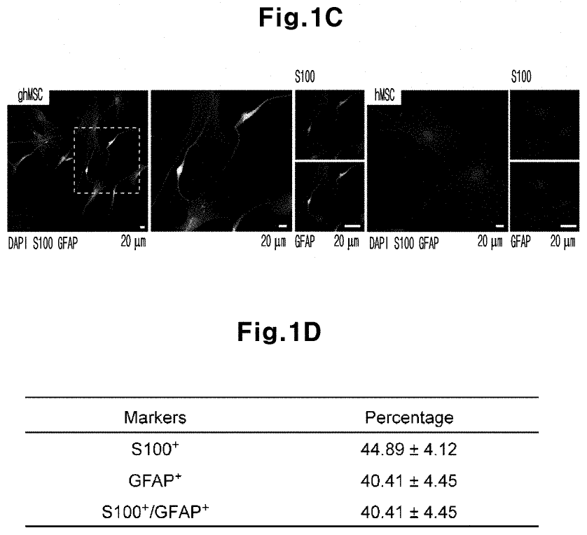

In a specific embodiment of the present invention, glia-like cells induced from human hMSCs (ghMSCs) were changed in shape (see FIG. 1a), which confirmed that they showed molecular characteristics of astrocytes and oligodendrocytes (see FIG. 1b). It was confirmed that astrocyte markers (GFAP, S100β and SLC1A3), Schwann cell/oligodendrocyte marker (Sox10) and oligodendrocyte progenitor cell marker (PDGFRα) and related genes were increased in ghMSCs compared to hMSCs.

It was confirmed that the genes related to astrocyte markers (GFAP, S100β and SLC1A3), Schwann cell/oligodendrocyte marker (Sox10) and oligodendrocyte progenitor cell marker (PDGFRα) were increased in ghMSCs compared to hMSCs.

In addition, it was confirmed that the mRNA expression of IGFBP-4, IGF-1, PAPP-A (pregnancy-associated plasma protein A), and protease, which play an important role in the ischemic condition, was increased in glia-like cells (see FIGS. 1c and 1d).

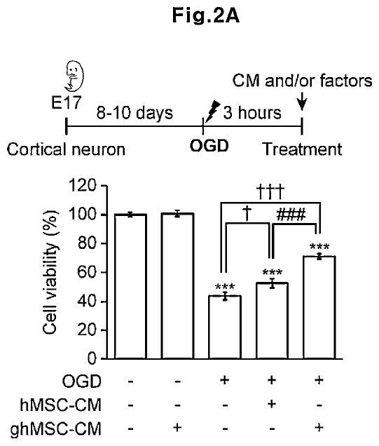

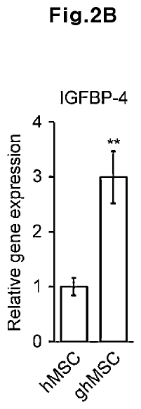

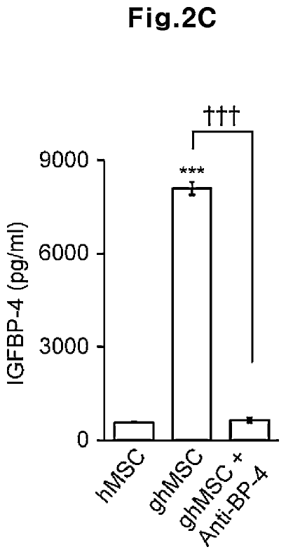

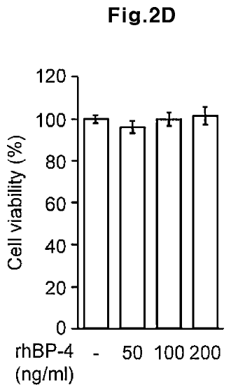

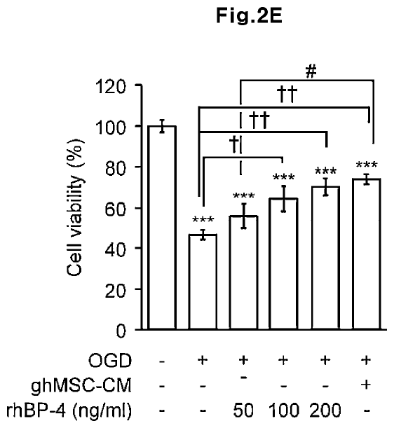

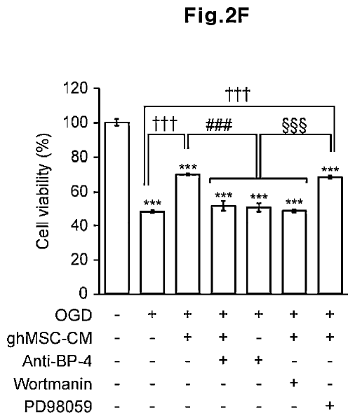

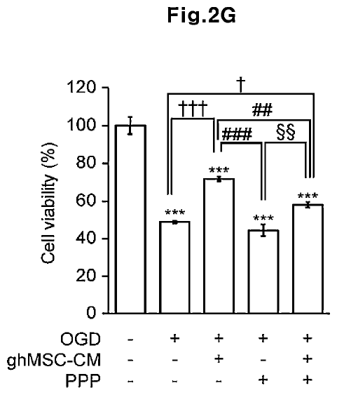

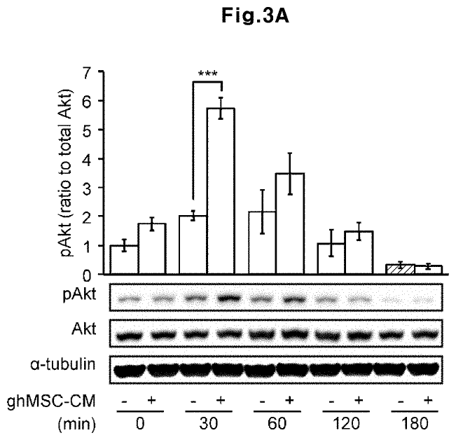

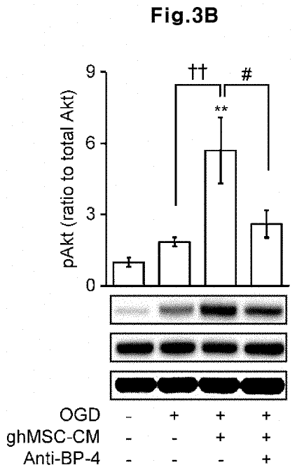

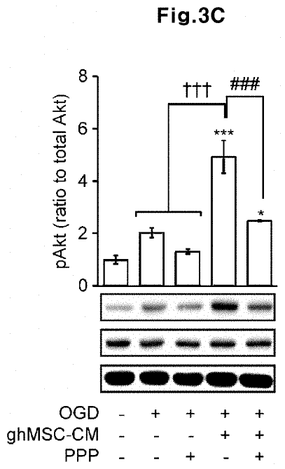

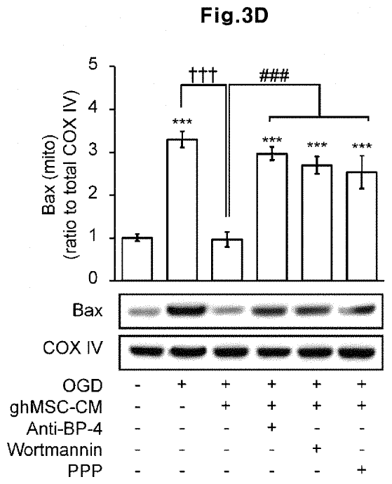

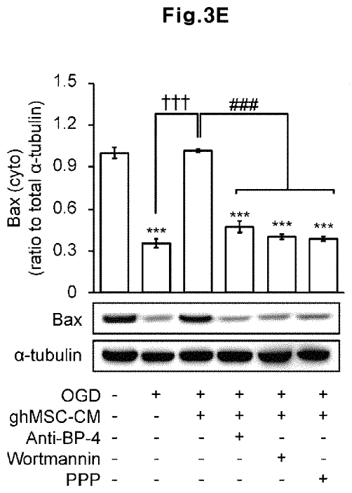

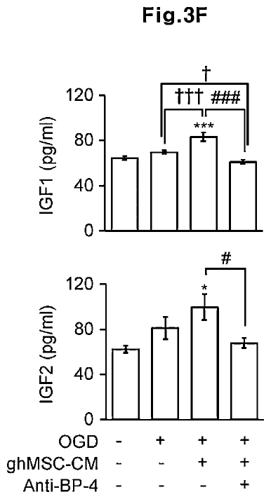

It was also confirmed that when the conditioned medium of late-passage human mesenchymal stem cells was treated in the culture medium in which the primary cortical neurons of oxygen-glucose deprivation (OGD) were concentrated, the viability of neurons was increased, and the neuroprotective effect was exhibited by overexpressing IGFBP-4 (see FIG. 2). When the conditioned medium of late-passage human mesenchymal stem cells was treated in the culture medium in which the primary cortical neurons of oxygen-glucose deprivation (OGD) were concentrated, the phosphorylation of Akt was increased. However, treatment of anti-BP-4 significantly reduced the phosphorylation of Akt, which had been increased. It was confirmed that this neuroprotective mechanism is through the Akt pathway of IGFBP-4 via IGF-1R (see FIGS. 3a to 3c). When the conditioned medium of late-passage human mesenchymal stem cells was treated in the culture medium in which the primary cortical neurons of oxygen-glucose deprivation (OGD) were concentrated, the expression of the proapoptotic protein Bax in mitochondria was increased and the expression thereof in cytoplasm was decreased. These results indicate that the Akt activity induced by IGFBP-4 reduces the endogenous Bax migration from cytoplasm to mitochondria and increases the neuronal survival by regulating the expressions of extracellular IGF-1 and IGF-2 (see FIGS. 3d, 3e and 3f).

The present invention showed that the IGFBP-4 secreted by ghMSCs had a neuroprotective effect in an in vitro/in vivo ischemic stroke model. Late-passage hMSCs induced into ghMSCs released high amount of IGFBP-4 and were found to be suitable cells for treating ischemic stroke. In addition, it was confirmed that the mechanism of stroke treatment by ghMSCs was achieved through the IGFBP-4 released from cells with IGF-1R and PI3/Akt, or ERK signaling.

The levels of pAkt were significantly increased by the treatment and injection of ghMSCs in vitro/in vivo. However, these effects showed opposite results by the treatment and injection of anti-BP-4 or PPP. These results indicate that the IGFBP-4 secreted from ghMSCs enhances the phosphorylation of Akt by IGF-1R signaling. Activation of Akt prevented the migration of Bax into mitochondria, and thus inhibited apoptosis by mitochondria. This activity indicates that the mitochondrial transition of Bax due to oxygen-glucose deprivation was inhibited by IGFBP-4 in ghMSC-CM, and the expression of Bax itself was reduced by IGFBP-4 secreted from ghMSCs (FIGS. 3d and 3e).

IGF-1R-mediated apoptosis signaling is associated with extracellular IGF-1 and IGF-2, and their levels are regulated by IGFBP-4 secreted from ghMSCs. It was found that IGFBP-4 interacts with IGF-1 and IGF-2, binds to these proteins, and inhibits the activity of IGF (FIG. 3f).

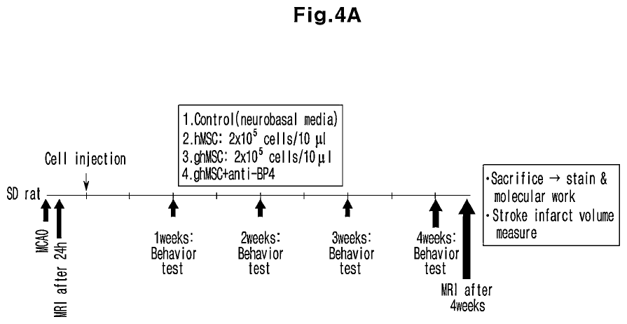

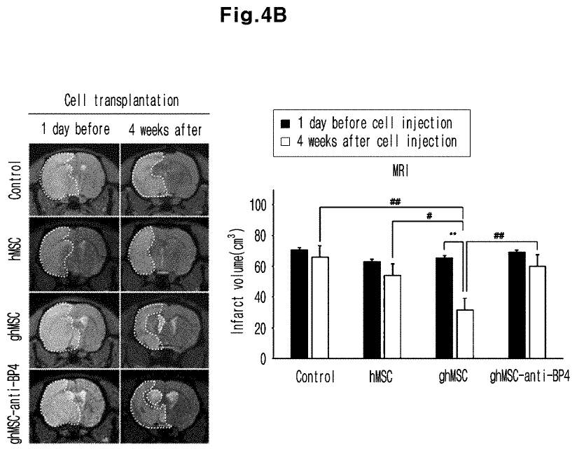

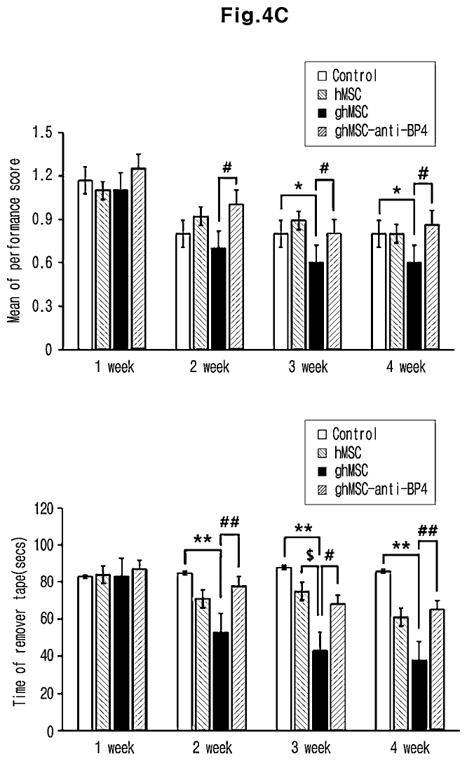

As a result of injecting glia-like cells into the infarction-induced animal model, the infarct volume was significantly reduced by more than 50%, and the neural function was significantly improved in the beam-walking test and sticky tape test compared to the control group and the group treated with human mesenchymal stem cells (hMSCs). It was confirmed that these results were due to the Akt pathway of IGFBP-4 via IGF-1R (see FIGS. 4b and 4c).

Therefore, the differentiated glia-like cells of the present invention can be effectively used as a cell therapy product for stroke by activating the Akt pathway via IGF-1R by hypersecreting the IGFBP-4 contained therein.

The composition for cell therapy of the present invention is preferably a pharmaceutical composition, and it can be formulated by the conventional method well known to those in the art. For example, the composition can be formulated as injectable solutions for parenteral administration such as sterilized solutions or suspensions by mixing with water or other pharmaceutically acceptable liquids. Particularly, the composition can be mixed with pharmaceutically acceptable carriers or mediums, such as sterilized water, saline, vegetable oil, emulsifiers, suspensions, surfactants, stabilizers, excipients, vehicles, antiseptics and binders, and then formulated as a unit dosage authorized by manufacture of medicines. The effective dose of the composition of the present invention means the dose inducing proper amount within designated conditions. Sterile composition for the injection can be provided by using an excipient such as distilled water for injection. To prepare injectable solutions, isotonic solution including saline, glucose or other adjuvants, which is exemplified by D-sorbitol, D-mannose, D-mannitol, and sodium chloride can be co-used with alcohol, particularly ethanol, polyalcohol such as propylene glycol, polyethylene glycol and non-ionic surfactant polysorbate 80 (TM), HCO-50. The oil is exemplified by sesame oil and soybean oil, which can be used together with such solubilizing agents as benzyl benzoate and benzyl alcohol. The oil can also be mixed with buffers such as phosphate buffer, sodium acetate buffer; soothing agents such as procaine hydrochloride; stabilizers such as benzyl alcohol and phenol; and antioxidants. The prepared injectable solution is filled in proper ampoules.

The preferable administration method to a patient is parenteral administration. Specifically, one-time administration to the injured site is basic, but multiple administrations are also acceptable. Administration time can be short or long. Examples of formulations for preferred administration are injections and percutaneously administrable preparations.

The effective dose of the composition is 10³˜10⁹ cells/kg, preferably 10⁴˜10⁸ cells/kg, and more preferably 6×10⁵˜6×10⁷ cells/kg. The composition may be administered 2˜3 times a day. The above conditions are not necessarily limited thereto, and may vary depending on the patient's condition and the degree of onset of disease.

Hereinafter, the present invention will be described in detail by the following examples and experimental examples.

However, the following examples and experimental examples are only for illustrating the present invention, and the contents of the present invention are not limited thereto.

### Example 1: Culture of Human Mesenchymal Stem Cells (hMSCs)

Adult human mesenchymal stem cells (hMSCs, Cambrex Bioscience, Walkersville, Md., USA) extracted from normal human bone marrow were cultured in low-glucose Dulbecco's modified Eagle's medium (DMEM) supplemented with 10% fetal bovine serum (FBS; Gibco, Waltham, Mass., Mass., USA). Cells (passages 6-12) were passaged 12 to 15 times in an environment of 37° C. and 5% CO₂ to obtain late-passage hMSCs.

### Example 2: Induction of Human Mesenchymal Stem Cells (hMSCs) into Glia-Like Cells (ghMSCs)

The late-passage human mesenchymal stem cells obtained in Example 1 were cultured for 24 hours in the primary differentiation medium (DMEM-low-glucose medium, 10% FBS, 1% penicillin-streptomycin mixture, 1 mM β-mercaptoethanol). Then, the human mesenchymal stem cells were washed with PBS (phosphate buffered solution) and cultured in the secondary differentiation medium [DMEM-low-glucose medium, 10% FBS, 1% penicillin-streptomycin mixture, 0.28 μg/ml of tretinoin (all-trans-retinoic acid)]. After 3 days, the mesenchymal stem cells were washed with PBS and then cultured in the tertiary differentiation medium (DMEM-low-glucose medium, 10% FBS, 1% penicillin-streptomycin mixture, 10 μM forskolin, 10 ng/ml of human basic-fibroblast growth factor, 5 ng/ml of human platelet derived growth factor-AA, 200 ng/ml of heregulin-β1) for 8 days. At this time, the tertiary differentiation medium was replaced once every two days. The morphology of the hMSCs in culture for the induction of differentiation was observed under a microscope. Cell survival during the induction of differentiation was observed through bright-field imaging (FIG. 1a).

### Example 3: Preparation of Glia-Like Cell-Enriched Conditioned Medium (ghMSC-CM)

Glia-like cells (1×10⁴ cells/cm²) were rinsed twice with PBS and cultured in serum-free Neurobasal-A medium (NB medium) for 18 hours. The medium was collected and cetrifuged at 1500 g for 5 minutes to remove cell debris, and then used for the experiments.

### Example 4: Freezing and Thawing of Glia-Like Cells (ghMSCs)

### <4-1> Freezing of Glia-Like Cells

A frozen vial was prepared by adjusting the total volume to 1 ml by mixing 1˜2×10⁵ human mesenchymal stem cells (or glia-like cells) in DMEM containing 10% fetal bovine serum (FBS), FBS stock solution, and DMSO in a ratio of 5:4:1, respectively. The prepared frozen vial was placed in a freezing container containing 100% isopropanol. The prepared vial was stored overnight in a −80° C. DEEP freezer, and then stored in a liquid nitrogen tank (LN2 tank).

### <4-2> Thawing of Glia-Like Cells

The frozen vial stored in the liquid nitrogen tank was thawed in a 37° C. water bath for about 2 minutes, and the cells in the frozen vial with a little ice were mixed well using a pipette in a biosafety hood. 1 ml of the cell concentrate in the frozen vial was mixed with 10 ml of DMEM containing 10% FBS and placed in a 15 me conical tube. The cell pellet was precipitated by centrifugation at 1,200 rpm for 5 minutes, followed by sucking all of the supernatant. 1 me of DMEM containing 10% fetal bovine serum (FBS) was placed in the 15 ml conical tube, and the cell pellet was suspended using a pipette. 11 μl of the cell pellet was mixed with 11 μl of trypan blue. 10 μl of the mixture was injected into a cell counter to calculate the number of cells contained in 1 ml, and plated on a cell culture dish at the density of 2,000 cells/cm².

### Experimental Example 1: Characterization of Glia-Like Cells (ghMSCs) Derived from Late-Passage Human Mesenchymal Stem Cells

Glia-like cells were induced from human mesenchymal stem cells by the method of Example 2. Inducing glia-like cells and induced glia-like cells were frozen and thawed, and then used for analysis. To confirm the characteristics of the induced glia-like cells, the expression levels of glia markers were confirmed by quantitative RT-PCR and immunohistochemical staining.

Particularly, for quantitative RT-PCR, RNA was extracted using TRIzol reagent (Invitrogen, Carlsbad, Calif., USA) and treated with DNase. For cDNA synthesis, reverse transcription was performed at 42° C. for 1 hour using M-MLV reverse transcriptase (Promega). SYBR FAST qPCR Kits (KAPA Biosystems) were used to identify the genes expressed in cDNA with the respective gene-specific primers represented by SEQ. ID. NO: 1˜NO: 24. The expression level of the target gene was confirmed based on GAPDH using a Ct method. The ΔCt value is the value minus the Ct value, and the cycle number of the critical value was measured as follows: ΔCt=Ct(Target)−Ct(GAPDH). The relative value of the target gene to endogenous GAPDH was determined by fold-change of GAPDH=2^{-ΔCt}.

In addition, for immunohistochemical staining, the cultured cells were fixed with 4% paraformaldehyde (PFA), and cultured with a blocking solution containing 5% standard goat serum and 0.1% Triton-X100. The cells were stained with GFAP (1:200; Merck Millipore) and S100 (1:250; Dako) for one day in a 4° C. refrigerator. The staining solution was washed with PBS several times, and the cells were stained with the secondary antibodies Alexa Fluor®488 anti-mouse IgG (Molecular Probes) and Alexa Fluor®546 anti-rabbit IgG (Molecular Probes) for 1 hour at room temperature. Then, the nuclei were stained with 4,6-diamidino-2-phenylindole (DAPI) (Santa Cruz Biotechnology). The samples were observed with a digital inverted fluorescence microscope (DM5000B; Leica).

As a result, as shown in FIG. 1b, it was confirmed that the expression levels of the radial glia markers Nestin, Sox2, Pax6, Vimentin and GFAP; the astrocyte markers GFAP, S100β and SLC1A3; and the oligodendrocyte marker Sox10 were increased in ghMSCs than in hMSCs. It was also confirmed that the expression levels of IGFBP-4 and IGF-1 pregnancy-associated plasma protein-A (PAPP-A), which play important roles in ischemic conditions, were increased in ghMSCs than in hMSCs (FIG. 1b). In addition, as shown in FIGS. 1c and 1d, it was confirmed that Sox10 and GFAP, the oligodendrocyte markers, were expressed in 45% and 40% of total glia-like cells (ghMSCs), respectively. The above results suggest that the glia-like cells derived from human mesenchymal stem cells contain a lot of astrocytes and oligodendrocytes that help the growth of surrounding cells and improve the surrounding environment, so these cells have the potential to treat stroke.

Experimental Example 2: Confirmation of Neuroprotective Effect of Glia-Like Cells on Primary Cortical Neurons Treated with Oxygen-Glucose Deprivation (OGD)

The neuroprotective effect of glia-like cells differentiated from late-passage human mesenchymal stem cells was confirmed as follows.

### 20<2-1> Preparation of Primary Cortical Neuron-Enriched Culture Medium and Oxygen Glucose Deprivation (OGD) Treatment for Primary Cortical Neurons

All the animal-related procedures were approved by the Seoul National University Institutional Animal Care and Use Committee (Seoul, Republic of Korea). A primary cortical neuron-enriched culture medium was prepared according to the method described above. Briefly, cerebral cortex was obtained from E17 SD rat embryos. The isolated cells in Neurobasal-A medium (NB; Gibco) supplemented with 10% fetal bovine serum (FBS) were plated at the density of 7.5×10⁴ cells/cm² on a plate coated with 50 μg/ml of poly-D-lysine (PDL) and 5 μg/ml of laminin. This culture medium was replaced with NB medium supplemented with 2% B27 (Gibco) after 24 hours. The medium was changed every 3 or 4 days to obtain the primary cortical neuron-enriched culture medium.

To induce oxygen-glucose deprivation (OGD), cortical neurons were rinsed twice with phosphate-buffered saline (PBS; pH 7.4) and 95% nitrogen gas, and 5% Co₂ gas mixture-injected glucose-free DMEM (OGD medium) was added thereto. The cells were placed in a humidified room, supplied with deoxidized gas (95% N₂, 5% CO₂) and stored in a 37° C. incubator for 3 hours. OGD was terminated by changing the culture medium, followed by reoxygenation with 5% Co₂ in a 37° C. incubator for 24 hours. The apoptosis induction model through oxygen-glucose deprivation (OGD) sets up an environment similar to the ischemic state caused by the interruption of blood supply, and induces apoptosis of brain neurons according to the ischemic state.

### <2-2> Confirmation of Neuroprotective Effect of Glia-Like Cell-Enriched Conditioned Medium

The neuroprotective effect of human mesenchymal stem cells and glia-like cells differentiated therefrom was confirmed using the oxygen-glucose deprivation (OGD)-induced primary cortical neuron-enriched culture medium of Experimental Example 2-1.

Particularly, human mesenchymal stem cells and glia-like cells (1×10⁴ cells/cm²) were rinsed twice with PBS and cultured in serum-free NB medium for 18 hours. The collected culture medium was added to the oxygen-glucose deprivation (OGD)-treated primary cortical neuron-enriched culture medium.

The glia-like cell-enriched conditioned medium (ghMSC-CM) was treated with 40 μg/ml IGFBP-4 antibody (anti-BP-4; R&D Systems), 50 μM PD98059 (Promega Corp, Madison, Wis., USA), 100 nM wortmannin (Sigma-Aldrich), 500 nM picropodophyllin (PPP; Santa Cruz Biotechnology, Santa Cruz, Calif., USA), or recombinant human IGFBP-4 (rhBP-4; PeproTech) for 15, 30, 15 or 30 minutes, respectively, followed by pre-culture. The OGD-treated primary cortical neuron-enriched culture medium was incubated in the glia-like cell-enriched conditioned medium (ghMSC-CM) prepared above.

Quantitative RT-PCR was performed in the same manner and conditions as in Experimental Example 1, except that the IGFBP-4 specific primers represented by SEQ. ID. NO: 25 and SEQ. ID. NO: 26 were used.

Cell viability was assessed using MTT assay. MTT reagent was added to the neurons cultured in a 4-well plate at the final concentration of 1 mg/ml, and cultured at 37° C. for 1 hour. MTT solution was carefully aspirated and formazan crystals were dissolved in 130 μl of DMSO. Absorbance was measured at 554 nm with a microplate reader.

For ELISA, the plate was washed twice with PBS, and additionally treated in Neurobasal media without serum protein according to the experimental conditions and cultured for 30 hours. Centrifugation was performed at 1200 rpm for 5 minutes to obtain supernatant. The supernatant was analyzed using IGFBP-4 DuoSet ELISA for human (R&D systems). The optimal density was measured with a microplate reader (Epoch2, Bio-Tek, Winooski, Vt., USA) at 450 nm.

As a result, as shown in FIG. 2a, it was confirmed that the cell viability was increased when the glia-like cell-enriched conditioned medium was treated than when the human mesenchymal stem cell-enriched conditioned medium. In addition, as shown in FIGS. 2b and 2c, it was confirmed that IGFBP-4 was expressed more than three times in glia-like cells than in human mesenchymal stem cells. As shown in FIGS. 2d and 2e, IGFBP-4 itself showed neuroprotective effects. These results indicate that the glia-like cells derived from human mesenchymal stem cells overexpress IGFBP-4 and thus exhibit neuroprotective effects.

### <2-3> Confirmation of Neuroprotective Effect of IGFBP-4

In order to confirm the mechanism of the neuroprotective effect of IGFBP-4, an inhibitor of PI3K/IGF and ERK signaling mechanism was treated, and the cell viability was measured in the same manner and conditions as in Experimental Example 2-2.

To investigate the IGFBP-4-mediated signaling showing neuroprotective effects by ghMSC-CM, two different signaling pathways related to IGF, phosphoinositide 3-kinase(PI3K)/Akt and extracellular signal-regulated kinase (ERK) pathways, were identified. Wortmannin, a PI3K/Akt inhibitor, abrogated the neuroprotective effect of the OGD-treated primary cortical neuron-enriched culture medium (FIG. 2f). However, there was no effect when the ERK inhibitor PD98059 was treated (FIG. 2f). The PI3K/Akt pathway is involved in IGF-1 receptor (IGF-1R) signaling, and the IGF-1R inhibitor PPP reduced the neuroprotective effect of ghMSC-CM (FIG. 2g). These results suggest that the IGF1R pathway via IGFBP-4 plays an important role in neuronal protection in hypoxia-injured primary cortical neurons.

### Experimental Example 3: Confirmation of IGFBP-4-Mediated Signaling Mechanism in Glia-Like Cell-Enriched Conditioned Medium

### <3-1> Confirmation of Akt Activation by IGFBP-4 and IGF-1R in Glia-Like Cell-Enriched Conditioned Medium

To confirm the Akt activation in the IGFBP-4 mediated neuroprotective mechanism, the degree of the Akt phosphorylation induced by the glia-like cell-enriched conditioned medium (ghMSC-CM) was measured at different time points after OGD treatment.

Particularly, cells were washed twice with cold PBS and cultured in RIPA buffer (50 mM Tris-HCl, pH 7.4, 1% NP40, 0.25% sodium deoxycholate, 150 mM NaCl, 1 mM Na₃VO₄, 0.2 mg/ml of leupeptin, 0.2 mg/ml of aprotinin, 0.1 M PMSF, and 0.5 M sodium fluoride). The separated supernatant was diluted in 3% BSA blocking solution at a ratio of 1:1000 with Akt (Cell Signaling, Beverly, Mass., USA), pAkt (Cell Signaling) and α-tubulin (Sigma Aldrich) as the primary SDS-PAGE antibodies. After incubating thereof with a goat-anti-rabbit or goat-anti-mouse HRP-conjugated secondary antibody (1:5000; Sigma-Aldrich), the antigen to be detected was detected with a Western blot detection kit (Thermo Fisher Scientific, Inc.). Mitochondria and cytoplasmic fragments were isolated with a mitochondrial fraction kit. (Active Motif, Carlsbad, Calif., USA).

As a result, the Akt phosphorylation was significantly increased when the glia-like cell-enriched condtioned medium (ghMSC-CM) was treated for 30 minutes (FIG. 3a). After ghMSC-CM treatment, the degree of the phosphorylated Akt (pAkt) was confirmed with or without OGD treatment and anti-IGFBP-4 antibody treatment. The significantly increased pAkt (P<0.01) incubated with ghMSC-CM after OGD treatment was significantly reduced by anti-BP-4 (FIG. 3b). In addition, the ghMSC-CM first incubated with the IGF-1R inhibitor PPP significantly reduced the expression of pAkt increased by ghMSC-CM (FIG. 3c). These results suggest that the Akt pathway-inducing activity of IGFBP-4 via IGF-1R plays a major role in the neuroprotective effect mediated by glia-like cells on OGD-injured primary cortical neurons.

### <3-2> Confirmation of Mitochondrial Bax Level in Glia-Like Cell-Enriched Conditioned Medium

In cortical neurons, oxygen-glucose deprivation (OGD) increased the level of mitochondrial Bax and decreased the level of cytoplasmic Bax, a pro-apoptotic protein (FIGS. 3d and 3e). In order to confirm the apoptosis inhibitory effect of glia-like cells, Western blotting was performed in the same manner and conditions as in Experimental Example 3-1, except that the primary antibodies of Bax (BD Pharmingen, San Jose, Calif., USA) and COX IV (Cell Signaling) were used to measure the expression level of the protein.

As a result, treatment of glia-like cells prevented Bax translocation induced by apoptosis and significantly restored the OGD-induced mitochondrial and cytoplasmic Bax expression to the control levels (FIGS. 3d and 3e). However, the effect of treatment of glia-like cells was decreased due to IGFBP-4 inhibition by anti-BP-4, Akt inhibition by wortmannin, and IGF-1R inhibition by PPP, involved in the neuroprotective signaling pathway of IGF-1-dependent IGFBP-4. This inhibition brought the expression level of mitochondrial Bax to the level at the time of OGD treatment, and returned the reduced cytoplasmic Bax to the value at the time of OGD treatment (FIGS. 3d and 3e).

These results suggest that the Akt activity induced by IGFBP-4 decreased the endogenous Bax migration from the cytoplasm to the mitochondria, thereby increasing neuronal survival.

### <3-3> Confirmation of Regulation of Extracellular IGF-1 and IGF-2 Expressions in Glia-Like Cell-Enriched Conditioned Medium

IGFBP-4 has a high affinity to IGF-1 and IGF-2, and IGFBP-4 regulates the activities of IGF-1 and IGF-2 in vitro/in vivo. Thus, whether IGFBP-4 regulates the expressions of extracellular IGF-1 and IGF-2 in OGD-treated primary cortical neurons was confirmed in the same manner and conditions as in Experimental Example 2-2 using ELISA.

As a result, as shown in FIG. 3f, the OGD treatment did not alter the expressions of extracellular IGF-1 and IGF-2. However, the treatment of glia-like cells after the OGD treatment significantly increased the expression of extracellular IGF-1 compared to the control and OGD-treated groups. This increase was reversed by the treatment of anti-IGFBP-4 antibody. Similarly, the treatment of glia-like cells after the OGD treatment increased the extracellular expression of IGF-2 compared to the control group in which the expression was decreased sue to IGFBP-4 inhibition (P<0.05). These results suggest that the IGFBP-4 highly expressed in glia-like cells plays a major role in regulating the expressions of extracellular IGF-1 and IGF-2 in the neuroprotective process to OGD treatment-induced primary cortical neuron damage.

### Experimental Example 4: Confirmation of Neuroprotective Effect of Glia-Like Cells on Cerebral Infarction

### <4-1> Inducing Temporary Infarction in Experimental Animals

To induce infarction, experimental animals were subjected to transient convulsions for 2 hours.

Particularly, male Sprague-Dawley rats (Orient Bio, Seoul, Korea) weighing 270˜300 g were used for the experiment. After adaptation and pre-training for neurological examination for 7 days, the left middle cerebral artery (MCA) of the SD rats was occluded for 2 hours using an intracerebral filament technique. The rats were anesthetized with isoflurane mixed with 30% oxygen and 70% nitrous oxide. The body temperature was maintained at 36.6±0.5° C. by a thermistor-controlled heating pad. Arterial pH, pCO₂, pO₂, and hematocrit were measured with 0.1 ml of arterial blood obtained from the right femoral catheter using a blood analysis system (International Technidyne, Edison, N.J., USA). Arterial pressure was monitored with a femoral catheter, and phase pressure, mean arterial pressure (MAP), and heart rate (HR) were recorded at a sampling rate of 200/s using a data acquisition system and laboratory computer (MacLab Bridge Amplifier, AD Instruments Pty Ltd., Castle Hill, Australia). Two hours after the occlusion, reperfusion was performed. Sham surgery was performed by inserting a thread into the left common carotid artery and then withdrawing it immediately. Other sham surgical procedures were the same as for transient MCAo. The ischemic core of caudate and putamen was evaluated by measuring the local cerebral blood flow (rCBF) using a laser Doppler flowmeter (ALF21; Advance, Tokyo, Japan) and a wire-type probe (0.3 mm in diameter; Unique Medical, Tokyo, Japan) inserted through a small-burr hole on the side of only 2 mm to the tomography of the cortical surface. 24 hours after the transient MCA occlusion, the presence of infarction was assessed by MRI.

### <4-2> Confirmation of Therapeutic Effect of Glia-Like Cell Transplantation on Infarction in Infarction-Induced Rat Model

To measure the therapeutic effect of glia-like cell transplantation on infarction, the rats were sacrificed 1 day and 4 weeks after infarction was induced, and the brains were imaged by MRI. And long-term neurological behavioral outcomes were evaluated.

Particularly, to measure the therapeutic effect of glia-like cell transplantation on infarction, the infarction SD rat model was divided into 4 groups. The first group was used as a control group, the second group was treated with hMSCs, the third group was treated with glia-like cells, and the last group was treated with glia-like cells and anti-BP4 antibody (100 μg/ml) together. Finally, 200,000 cells were placed in 10 [symbol] of NB medium and transplanted to the side of the rat brain.

All MRI images were obtained using a 3-Tesla MR machine with a ds microscope coil of 47 mm. The rats were sacrificed, and their brains were used for molecular biological measurements.

Neurobehavioral functions were evaluated weekly for 4 groups of the rats using the beam-walking test and the sticky-tape test for the neurological results. These evaluations were performed by investigators ignorant of the experimental groups before the transient middle cerebral artery occlusion (MCAo) and 1, 2, 3, and 4 weeks after the cell transplantation.

For the beam walking test, rats were trained to walk on a wooden beam 3 days before the middle cerebral artery occlusion (MCAo). The beam was installed at a distance of 60 cm from the floor, and the beam was installed for the rats to return to their home. Experimental results were scored as follows. 0: crossed the beam without slipping of the foot, 1: grasped the side of the beam and crossed the beam, 2: crossed, but seemed impossible to walk on the beam, 3: took a considerable amount of time to cross the beam due to difficulty walking, 4: impossible to cross the beam, 5: impossible to move body or limbs on the beam, and 6: impossible to stay on the beam for more than 10 seconds.

The modified sticky tape test was performed by making a sleeve using a 3 cm piece of green paper tape. A 1 cm wide tape was wrapped around the forefoot to allow the tape to self-adhere and the fingers to protrude slightly from the sleeve. A typical response of rats is to use the mouth to pull the tape to remove it, or to rub it with the opposite paw to release the tape. After attaching the tape, the rats were placed in cages and observed for 30 seconds. A timer was turned on while the rats tried to remove the tape. The test was repeated three times on each test day, and the highest two scores were averaged. The exercise test data showed the mean value of duration and right/left ratio.

As a result, as shown in FIG. 4b, it was confirmed by MRI that the infarct volume was significantly reduced by more than 50% by the injection of glia-like cells (ghMSCs) one day after the infarction induction. The effect of glia-like cells (ghMSCs) was about 20% higher than that of human mesenchymal stem cells (hMSCs). This effect was almost blocked by anti-BP-4 antibody.

In addition, as shown in FIG. 4c, the neuronal function was significantly improved in the group treated with glia-like cells (ghMSCs). In the beam-walking test, the treatment with glia-like cells (ghMSCs) dramatically lowered the average performance score showing neurobehavioral improvement to 0.6 points at 3 weeks after the cell injection, and this change was blocked by anti-BP-4 antibody. In the sticky-tape test, the time taken to remove sticky tape from the limb was reduced to 40 seconds in the rats treated with glia-like cells (ghMSCs), but this decrease was disappeared by the treatment of anti-BP-4 antibody.

### Experimental Example 5: Confirmation of IGFBP-4-Mediated Activation of PI3K/Akt Pathway in Brain Protected by ghMSCs in In Vivo Model

In Experimental Example 3, it was confirmed that the PI3K/Akt pathway was activated by IGFBP-4 by glia-like cells in vitro. Accordingly, it was confirmed whether the PI3K/Akt pathway was activated by IGFBP-4 by glia-like cells in an in vivo rat model.

Five rats for each of the four groups used in Experimental Example 4 were used for immunohistochemical staining analysis. The rat brain perfused with PBS was perfused again with 4% paraformaldehyde (PFA). Then, the brain was quickly extracted, fixed in 4% paraformaldehyde (PFA) for one day, and incubated in 30% sugar solution for 3 days. The brain tissue was mounted using an optimal cutting temperature (OCT) compound (Leica, Wetzlar, Germany), and 20 μm-thick brain sections were prepared using a motorized cryostat (Leica, Wetzlar, Germany). Mito Silane-coated slides were treated with 3% hydrogen peroxide to block the peroxidase activity inside the brain tissue. The tissue was stained with pAKT (1:500), p-ERK (1:500), truncated caspase-3 (1:500; Cell signaling), Bcl-xl (1:250; Cell Signaling), Bax (1:100; Abcam), cytochrome C (1:500; Santa Cruz Biotechnology), and NeuN (1:500; Millipore, Mass., USA) diluted in 10% FBS blocking solution at 4° C. overnight. After washing the stained tissue several times with PBS, the residual primary antibodies were washed with 5% FBS. The tissue was reacted with the secondary antibody conjugated to tetramethylrhodamineisothiocyanate (TRITC) (Invitrogen, Carlsbad, Calif., USA) or fluorescein isothiocyanate (FITC) (Invitrogen). The tissue was mounted with DAPI mounting solution (Vector Laboratories, H-500), and the fluorescence was confirmed with a fluorescence microscope (Olympus, Center Valley, Pa., USA).

The rats in each group were euthanized, and the brain tissues were perfused with PBS and stored at −80° C. The frozen brain tissues were microdissected on cold plates. The tissues were homogenized with RIPA II cell lysis buffer (100 mM Na₃VO₄, 100 mM PMSF, 100 mM NaF and protease inhibitor) and Q55 sonicator (Qsonica, Melville, N.Y., USA). An equal amount (40 μg) of protein was separated by 12% SDS-PAGE for 25 minutes, and transferred to a nitrocellulose membrane for 1 hour and 30 minutes. Primary antibodies to Akt (1:500), ERK (1:500; Cell Signaling), NeuN (1:200; Abcam, UK), Bax (1:500; Cell Signaling), pAkt (1:200), p-ERK (1:500; Cell Signaling), and truncated caspase-9 (1:500; Cell Signaling) were diluted in 2% skim milk blocking solution. The membrane was incubated for one day, treated with the anti-rabbit or anti-mouse HRP-conjugated secondary antibody (1:2000, Amersham Pharmacia Biotech, Piscataway, N.J., USA), and the proteins were detected with solution A and solution B (West-Q Chemiluminescent Substrate Kit, GenDEPOT).

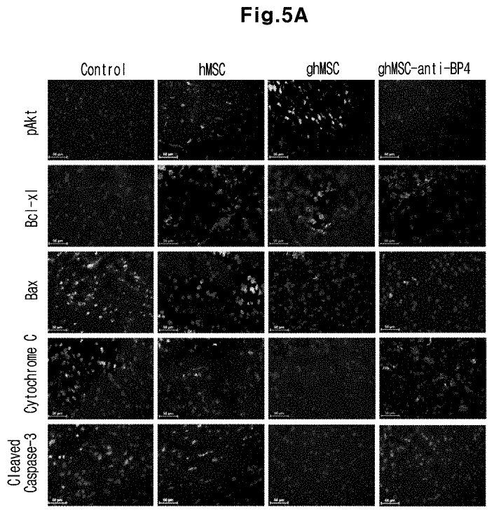

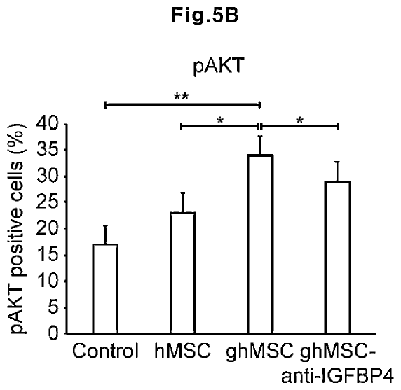

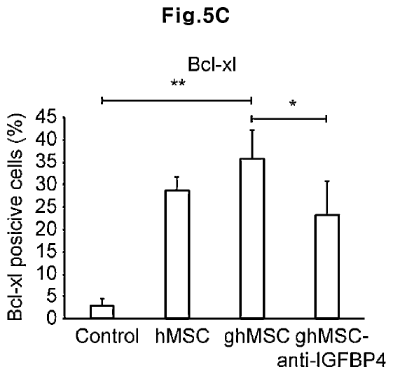

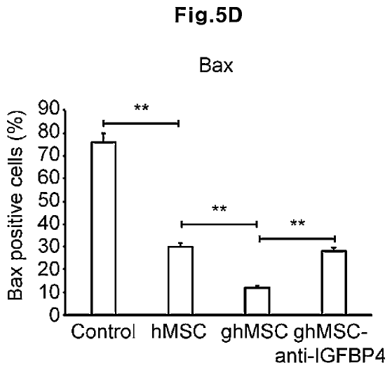

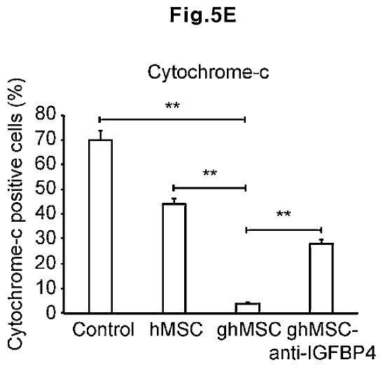

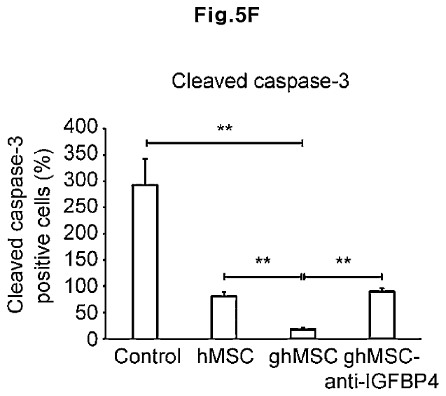

As a result, as shown in FIGS. 5a˜5f, the phosphorylated Akt (pAkt) and Bcl-xl were significantly increased more than two times in the brain injected with glia-like cells, but cytochrome c, Bax and truncated caspase-3 were significantly decreased to less than 10%. However, these changes induced by glia-like cells (ghMSCs) were significantly reduced by anti-BP-4 antibody (FIGS. 5a˜5f).

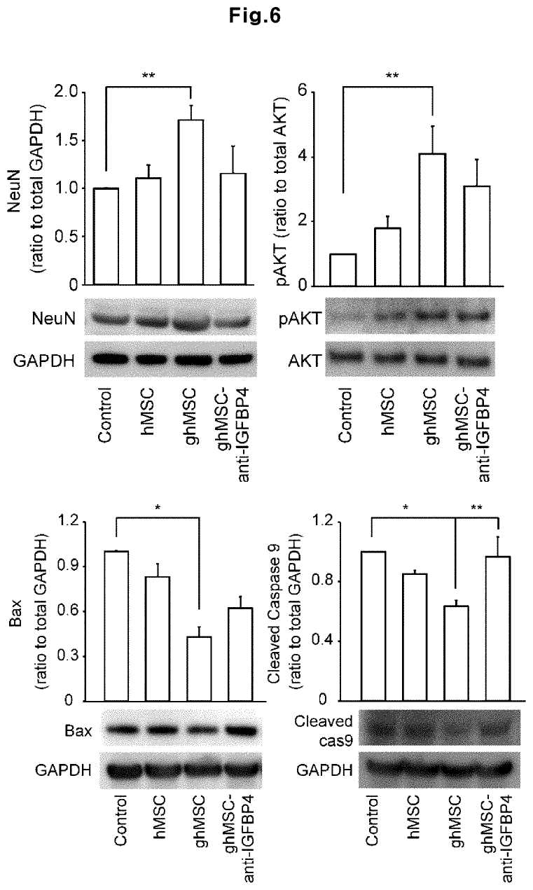

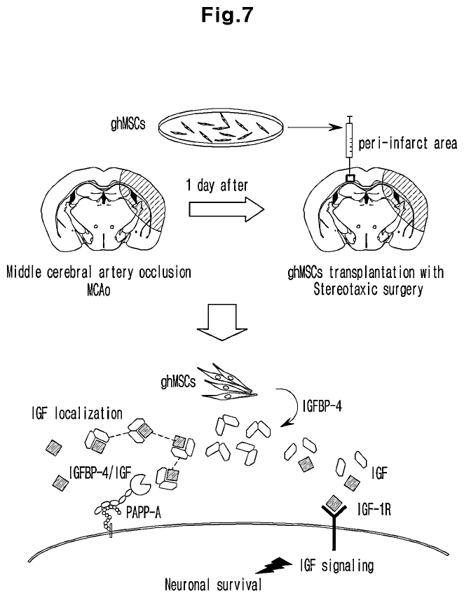

In addition, as shown in FIG. 6, it was confirmed by Western blotting that the expressions of NeuN (neuronal marker) and pAkt were increased more than two times in the brain transplanted with glia-like cells. And Bax and truncated caspase-9 were decreased according to the injection of glia-like cells (FIG. 6). This effect was reduced by the injection of anti-BP-4 antibody. These results indicate that IGFBP-4 plays a key role in the neuroprotective effect of glia-like cells on infarction.

1. A method for treating stroke in a subject comprising:
administering an effective amount of glia-like cells (ghMSCs) to the subject, wherein in the ghMSCs are induced from late-passage human mesenchymal stem cells (hMSCs),
thereby treating the subject.

2. The method for treating stroke according to claim 1, wherein the ghMSCs are oligodendroglia, astrocytes, microglia, radial glia, or a combination thereof.

3. The method for treating stroke according to claim 1, wherein the stroke is ischemic stroke or hemorrhagic stroke.

4. The method for treating stroke according to claim 1, wherein the late-passage hMSCs have been passaged 10 to 15 times.

5. The method for treating stroke according to claim 1, wherein the ghMSCs are derived from bone marrow, adipose tissue, blood, umbilical cord blood, liver, skin, gastrointestinal tract, placenta or uterus.

6. The method for treating a stroke according to claim 1, wherein the ghMSCs release IGFBP-4 (insulin-like growth factor binding protein-4).

7. The method for treating stroke according to claim 1, wherein the ghMSCs activate the PI3K/Akt pathway of IGFBP-4 via IGF-1R.

8. The method for treating stroke according to claim 1, wherein the ghMSCs are administered at a density of 6×10⁵˜6×10⁷ cells/kg.

9. The method for treating stroke according to claim 1, wherein the ghMSCs are adminstered as a cell therapy product.

10. The method for treating stroke according to claim 1, wherein the ghMSCs are administered parenterally.

11. A method for producing late-passage human mesenchymal stem cells (hMSCs) induced into glia-like cells (ghMSCs) comprising the following steps:
1) obtaining late-passage human mesenchymal stem cells by culturing human mesenchymal stem cells;
2) primary culturing the late-passage human mesenchymal stem cells obtained in step 1) for 22˜26 hours in DMEM containing 10% FBS and 1 mM β-mercaptoethanol;
3) secondary culturing the late-passage human mesenchymal stem cells for 68˜76 hours in DMEM containing 10% FBS and 0.28 μg/ml of tretinoin (all-trans-retinoic acid); and
4) tertiary culturing the late-passage human mesenchymal stem cells for 8 days in DMEM containing 10% FBS, 10 ng/ml of bFGF (basic fibroblast growth factor), 5 ng/ml of PDGF-AA, 10 μM Forskolin and 200 ng/ml of HRG-β1 (Heregulin-β1).

12. A composition for intra-individual IGFBP-4 (insulin-like growth factor binding protein-4) delivery comprising late-passage glia-like cells (ghMSCs) differentiated from human bone marrow-derived mesenchymal stem cells as an active ingredient.

13. A method for treating stroke comprising a step of:
administering late-passage human mesenchymal stem cells (hMSCs) induced into glia-like cells (ghMSCs) to a subject.


SEQUENCE LISTING (26 sequences)

SEQ ID NO: 1 - DNA, 20 bp, Artificial Sequence
  GAPDH forward primer
gtcagtggtggacctgacct

SEQ ID NO: 2 - DNA, 20 bp, Artificial Sequence
  GAPDH reverse primer
caccaccctgttgctgtagc

SEQ ID NO: 3 - DNA, 20 bp, Artificial Sequence
  GFAP forward primer
caacctgcagattcgagaaa

SEQ ID NO: 4 - DNA, 20 bp, Artificial Sequence
  GFAP reverse primer
gtcctgcctcacatcacatc

SEQ ID NO: 5 - DNA, 21 bp, Artificial Sequence
  Nestin forward primer
cacctgtgccagcctttctta

SEQ ID NO: 6 - DNA, 20 bp, Artificial Sequence
  Nestin reverse primer
tttcctcccaccctgtgtct

SEQ ID NO: 7 - DNA, 20 bp, Artificial Sequence
  Sox2 forward primer
agtctccaagcgacgaaaaa

SEQ ID NO: 8 - DNA, 20 bp, Artificial Sequence
  Sox2 reverse primer
gcaagaagcctctccttgaa

SEQ ID NO: 9 - DNA, 21 bp, Artificial Sequence
  Pax6 forward primer
aggtattacgagactggctcc

SEQ ID NO: 10 - DNA, 22 bp, Artificial Sequence
  Pax6 reverse primer
tcccgcttatactgggctattt

SEQ ID NO: 11 - DNA, 21 bp, Artificial Sequence
  Vimentin forward primer
agaactttgccgttgaagctg

SEQ ID NO: 12 - DNA, 23 bp, Artificial Sequence
  Vimentin reverse primer
ccagagggagtgaatccagatta

SEQ ID NO: 13 - DNA, 19 bp, Artificial Sequence
  sox10 forward primer
cggaccagtacccgcacct

SEQ ID NO: 14 - DNA, 21 bp, Artificial Sequence
  sox10 reverse primer
ggcgcttgtcactttcgttca

SEQ ID NO: 15 - DNA, 20 bp, Artificial Sequence
  S100beta forward primer
ggagacggcgaatgtgactt

SEQ ID NO: 16 - DNA, 23 bp, Artificial Sequence
  S100beta reverse primer
gaactcgtggcaggcagtagtaa

SEQ ID NO: 17 - DNA, 22 bp, Artificial Sequence
  PDGFR alpha forward primer
tacacttgctattacaaccaca

SEQ ID NO: 18 - DNA, 20 bp, Artificial Sequence
  PDGFR alpha reverse primer
atcctccacgatgactaaat

SEQ ID NO: 19 - DNA, 22 bp, Artificial Sequence
  SLC1A3 forward primer
ggttgctgcaagcactcatcac

SEQ ID NO: 20 - DNA, 22 bp, Artificial Sequence
  SLC1A3 reverse primer
cacgccattgttctcttccagg

SEQ ID NO: 21 - DNA, 22 bp, Artificial Sequence
  SLC1A2 forward primer
tgccaacagaggacatcagcct

SEQ ID NO: 22 - DNA, 22 bp, Artificial Sequence
  SLC1A2 reverse primer
cagctcagacttggagaggtga

SEQ ID NO: 23 - DNA, 21 bp, Artificial Sequence
  PAPP A forward primer
cagaatgcactgttacctgga

SEQ ID NO: 24 - DNA, 21 bp, Artificial Sequence
  PAPP A reverse primer
gctgatcccaattctctttca

SEQ ID NO: 25 - DNA, 20 bp, Artificial Sequence
  IGFBP4 forward primer
gcccaagaggactgagactg

SEQ ID NO: 26 - DNA, 20 bp, Artificial Sequence
  IGFBP4 reverse primer
cacatgccaaaatcagagga